SEQUENCE LISTING (43 sequences)

SEQ ID NO: 1 - DNA, 33 bp, Artificial Sequence
  source: /note=“Description of Artificial Sequence Synthetic primer”
taggatggccaatatttttggtggttggagcaa

SEQ ID NO: 2 - DNA, 39 bp, Artificial Sequence
  source: /note=“Description of Artificial Sequence Synthetic primer”
aagcttcgtgattaataaattaatggtggttggagcaga

SEQ ID NO: 3 - DNA, 43 bp, Artificial Sequence
  source: /note=“Description of Artificial Sequence Synthetic primer”
atcggacttcttaaaataataaattaattggttggagcaggtc

SEQ ID NO: 4 - DNA, 49 bp, Artificial Sequence
  source: /note=“Description of Artificial Sequence Synthetic primer”
aacttctgggatttaaaaatattttataatatatggttggagcaggtgc

SEQ ID NO: 5 - DNA, 54 bp, Artificial Sequence
  source: /note=“Description of Artificial Sequence Synthetic primer”
atctatataaaaaaattaaaaattaataaatattaataaggttggagcaggtga

SEQ ID NO: 6 - DNA, 59 bp, Artificial Sequence
  source: /note=“Description of Artificial Sequence Synthetic primer”
accatggtttttatttattaatattatttatttaatttttataaggttggagcaggtgt

SEQ ID NO: 7 - DNA, 63 bp, Artificial Sequence
  source: /note=“Description of Artificial Sequence Synthetic primer”
tgagttaccaaatatttatttataatataaaaattattaaaataatataggttggagcaggtt

SEQ ID NO: 8 - DNA, 20 bp, Artificial Sequence
  source: /note=“Description of Artificial Sequence Synthetic primer”
agacaggatcaggtcagcgg

SEQ ID NO: 9 - DNA, 36 bp, Artificial Sequence
  source: /note=“Description of Artificial Sequence Synthetic primer”
tcagaaggacaataaaaatactggatacagctggaa

SEQ ID NO: 10 - DNA, 42 bp, Artificial Sequence
  source: /note=“Description of Artificial Sequence Synthetic primer”
tggcagtaggataataatattaatactggatacagctggact

SEQ ID NO: 11 - DNA, 47 bp, Artificial Sequence
  source: /note=“Description of Artificial Sequence Synthetic primer”
tgtggagatttaataattttaatataaatactggatacagctggacg

SEQ ID NO: 12 - DNA, 53 bp, Artificial Sequence
  source: /note=“Description of Artificial Sequence Synthetic primer”
agaaggaccgattaattaaaatttttatatttatactggatacagctggacgg

SEQ ID NO: 13 - DNA, 58 bp, Artificial Sequence
  source: /note=“Description of Artificial Sequence Synthetic primer”
aaacgcacaataattaataataaaataatttaattatatactggatacagctggacac

SEQ ID NO: 14 - DNA, 27 bp, Artificial Sequence
  source: /note=“Description of Artificial Sequence Synthetic primer”
agatcatcctttcagagaaaataatgc

SEQ ID NO: 15 - DNA, 29 bp, Artificial Sequence
  source: /note=“Description of Artificial Sequence Synthetic primer”
gcatatcacattttggtctagctacagat

SEQ ID NO: 16 - DNA, 24 bp, Artificial Sequence
  source: /note=“Description of Artificial Sequence Synthetic primer”
ccgcattttggtctagctacagag

SEQ ID NO: 17 - DNA, 39 bp, Artificial Sequence
  source: /note=“Description of Artificial Sequence Synthetic primer”
catacatagatacatataaattttggtctagctacagaa

SEQ ID NO: 18 - DNA, 34 bp, Artificial Sequence
  source: /note=“Description of Artificial Sequence Synthetic primer”
catcatgatcaattgattttggtctagctacaaa

SEQ ID NO: 19 - DNA, 60 bp, Artificial Sequence
  source: /note=“Description of Artificial Sequence Synthetic primer”
taaattttaattttaaaaccatggtttcaagcttcataaattaatggtggttggagcaga

SEQ ID NO: 20 - DNA, 60 bp, Artificial Sequence
  source: /note=“Description of Artificial Sequence Synthetic primer”
ataaatggtattttaattttaaaaccatggtttctaggatggctttggtggttggagcaa

SEQ ID NO: 21 - DNA, 47 bp, Artificial Sequence
  source: /note=“Description of Artificial Sequence Synthetic primer”
taacttctgggattaaatattttataatatatggttggagcaggtgc

SEQ ID NO: 22 - DNA, 69 bp, Artificial Sequence
  source: /note=“Description of Artificial Sequence Synthetic primer”
atatattgagttaccaaatatttatttataatataaaaattattaaaataatataggttggagcaggtt

SEQ ID NO: 23 - DNA, 50 bp, Artificial Sequence
  source: /note=“Description of Artificial Sequence Synthetic primer”
gctatataaaattaaaaattaataaatattaataaggttggagcaggtga

SEQ ID NO: 24 - DNA, 54 bp, Artificial Sequence
  source: /note=“Description of Artificial Sequence Synthetic primer”
ataaaaccatggtttatcggacttcaataataaattaattggttggagcaggtc

SEQ ID NO: 25 - DNA, 61 bp, Artificial Sequence
  source: /note=“Description of Artificial Sequence Synthetic primer”
ataccatggtttttatttattaatattatttatttaatttttataaggttggagcaggtgt

SEQ ID NO: 26 - DNA, 64 bp, Artificial Sequence
  source: /note=“Description of Artificial Sequence Synthetic primer”
atattaaaacgcacaataattaataataaaataatttaattatatactggatacagctggacac

SEQ ID NO: 27 - DNA, 71 bp, Artificial Sequence
  source: /note=“Description of Artificial Sequence Synthetic primer”
ttattaaaacttatatcgaaataatattatattataatttatttattaatatactggatacagctggacat

SEQ ID NO: 28 - DNA, 38 bp, Artificial Sequence
  source: /note=“Description of Artificial Sequence Synthetic primer”
tatcagaaggacaataaaaatactggatacagctggaa

SEQ ID NO: 29 - DNA, 45 bp, Artificial Sequence
  source: /note=“Description of Artificial Sequence Synthetic primer”
aattggcagtaggataataatattaatactggatacagctggact

SEQ ID NO: 30 - DNA, 52 bp, Artificial Sequence
  source: /note=“Description of Artificial Sequence Synthetic primer”
taaattgtggagatttaataattttaatataaatactggatacagctggacg

SEQ ID NO: 31 - DNA, 59 bp, Artificial Sequence
  source: /note=“Description of Artificial Sequence Synthetic primer”
taaattagaaggaccgattaattaaaatttttatatttatactggatacagctggacgg

SEQ ID NO: 32 - DNA, 41 bp, Artificial Sequence
  source: /note=“Description of Artificial Sequence Synthetic primer”
taggatggccttattaatattagaggaagccttcgcctgtc

SEQ ID NO: 33 - DNA, 60 bp, Artificial Sequence
  source: /note=“Description of Artificial Sequence Synthetic primer”
taggatggcctaattaataataaaataatttaattattggattagctggattgtcagtgc

SEQ ID NO: 34 - DNA, 35 bp, Artificial Sequence
  source: /note=“Description of Artificial Sequence Synthetic primer”
aacagcagccattaccagacccatccattcccgtg

SEQ ID NO: 35 - DNA, 30 bp, Artificial Sequence
  source: /note=“Description of Artificial Sequence Synthetic primer”
aattatattttggaagaacccagggcagaa

SEQ ID NO: 36 - DNA, 40 bp, Artificial Sequence
  source: /note=“Description of Artificial Sequence Synthetic primer”
ctttgacagatttatggaatcccacacgggacgtttcaat

SEQ ID NO: 37 - DNA, 34 bp, Artificial Sequence
  source: /note=“Description of Artificial Sequence Synthetic primer”
atatattttatcaccgcctggttactgtgtcctg

SEQ ID NO: 38 - DNA, 31 bp, Artificial Sequence
  source: /note=“Description of Artificial Sequence Synthetic primer”
agttgagatatagctcctgctacttccccag

SEQ ID NO: 39 - DNA, 39 bp, Artificial Sequence
  source: /note=“Description of Artificial Sequence Synthetic primer”
acagtaataacttacataatcccccttgtcgtttgaggt

SEQ ID NO: 40 - DNA, 4454 bp, Homo sapiens
gaaacgtcccgtgtgggaggggcgggtctgggtgcggcctgccgcatgactcgtggttcggaggcccacgtggccggggcggggactcaggcgcctggggcgccgactgattacgtagcgggcggggccggaagtgccgctccttggtgggggctgttcatggcggttccggggtctccaacatttttcccggctgtggtcctaaatctgtccaaagcagaggcagtggagcttgaggttcttgctggtgtgaaatgactgagtacaaactggtggtggttggagcaggtggtgttgggaaaagcgcactgacaatccagctaatccagaaccactttgtagatgaatatgatcccaccatagaggattcttacagaaaacaagtggttatagatggtgaaacctgtttgttggacatactggatacagctggacaagaagagtacagtgccatgagagaccaatacatgaggacaggcgaaggcttcctctgtgtatttgccatcaataatagcaagtcatttgcggatattaacctctacagggagcagattaagcgagtaaaagactcggatgatgtacctatggtgctagtgggaaacaagtgtgatttgccaacaaggacagttgatacaaaacaagcccacgaactggccaagagttacgggattccattcattgaaacctcagccaagaccagacagggtgttgaagatgctttttacacactggtaagagaaatacgccagtaccgaatgaaaaaactcaacagcagtgatgatgggactcagggttgtatgggattgccatgtgtggtgatgtaacaagatacttttaaagttttgtcagaaaagagccactttcaagctgcactgacaccctggtcctgacttccctggaggagaagtattcctgttgctgtcttcagtctcacagagaagctcctgctacttccccagctctcagtagtttagtacaataatctctatttgagaagttctcagaataactacctcctcacttggctgtctgaccagagaatgcacctcttgttactccctgttatttttctgccctgggttcttccacagcacaaacacacctctgccaccccaggtttttcatctgaaaagcagttcatgtctgaaacagagaaccaaaccgcaaacgtgaaattctattgaaaacagtgtcttgagctctaaagtagcaactgctggtgattttttttttctttttactgttgaacttagaactatgctaatttttggagaaatgtcataaattactgttttgccaagaatatagttattattgctgtttggtttgtttataatgttatcggctctattctctaaactggcatctgctctagattcataaatacaaaaatgaatactgaattttgagtctatcctagtcttcacaactttgacgtaattaaatccaactttcacagtgaagtgcctttttcctagaagtggtttgtagacttcctttataatatttcagtggaatagatgtctcaaaaatccttatgcatgaaatgaatgtctgagatacgtctgtgacttatctaccattgaaggaaagctatatctatttgagagcagatgccattttgtacatgtatgaaattggttttccagaggcctgttttggggctttcccaggagaaagatgaaactgaaagcacatgaataatttcacttaataatttttacctaatctccacttttttcataggttactacctatacaatgtatgtaatttgtttcccctagcttactgataaacctaatattcaatgaacttccatttgtattcaaatttgtgtcataccagaaagctctacatttgcagatgttcaaatattgtaaaactttggtgcattgttatttaatagctgtgatcagtgattttcaaacctcaaatatagtatattaacaaattacattttcactgtatatcatggtatcttaatgatgtatataattgccttcaatccccttctcaccccaccctctacagcttcccccacagcaataggggcttgattatttcagttgagtaaagcatggtgctaatggaccagggtcacagtttcaaaacttgaacaatccagttagcatcacagagaaagaaattcttctgcatttgctcattgcaccagtaactccagctagtaattttgctaggtagctgcagttagccctgcaaggaaagaagaggtcagttagcacaaaccctttaccatgactggaaaactcagtatcacgtatttaaacatttttttttcttttagccatgtagaaactctaaattaagccaatattctcatttgagaatgaggatgtctcagctgagaaacgttttaaattctctttattcataatgttctttgaagggtttaaaacaagatgttgataaatctaagctgatgagtttgctcaaaacaggaagttgaaattgttgagacaggaatggaaaatataattaattgatacctatgaggatttggaggcttggcattttaatttgcagataataccctggtaattctcatgaaaaatagacttggataacttttgataaaagactaattccaaaatggccactttgttcctgtctttaatatctaaatacttactgaggtcctccatcttctatattatgaattttcatttattaagcaaatgtcatattaccttgaaattcagaagagaagaaacatatactgtgtccagagtataatgaacctgcagagttgtgcttcttactgctaattctgggagctttcacagtactgtcatcatttgtaaatggaaattctgcttttctgtttctgctccttctggagcagtgctactctgtaattttcctgaggcttatcacctcagtcatttcttttttaaatgtctgtgactggcagtgattctttttcttaaaaatctattaaatttgatgtcaaattagggagaaagatagttactcatcttgggctcttgtgccaatagcccttgtatgtatgtacttagagttttccaagtatgttctaagcacagaagtttctaaatggggccaaaattcagacttgagtatgttctttgaataccttaagaagttacaattagccgggcatggtggcccgtgcctgtagtcccagctacttgagaggctgaggcaggagaatcacttcaacccaggaggtggaggttacagtgagcagagatcgtgccactgcactccagcctgggtgacaagagagacttgtctccaaaaaaaaagttacacctaggtgtgaattttggcacaaaggagtgacaaacttatagttaaaagctgaataacttcagtgtggtataaaacgtggtttttaggctatgtttgtgattgctgaaaagaattctagtttacctcaaaatccttctctttccccaaattaagtgcctggccagctgtcataaattacatattccttttggtttttttaaaggttacatgttcaagagtgaaaataagatgttctgtctgaaggctaccatgccggatctgtaaatgaacctgttaaatgctgtatttgctccaacggcttactatagaatgttacttaatacaatatcatacttattacaatttttactataggagtgtaataggtaaaattaatctctattttagtgggcccatgtttagtctttcaccatcctttaaactgctgtgaatttttttgtcatgacttgaaagcaaggatagagaaacactttagagatatgtggggtttttttaccattccagagcttgtgagcataatcatatttgctttatatttatagtcatgaactcctaagttggcagctacaaccaagaaccaaaaaatggtgcgttctgcttcttgtaattcatctctgctaataaattataagaagcaaggaaaattagggaaaatattttatttggatggtttctataaacaagggactataattcttgtacattatttttcatctttgctgtttctttgagcagtctaatgtgccacacaattatctaaggtatttgttttctataagaattgttttaaaagtattcttgttaccagagtagttgtattatatttcaaaacgtaagatgatttttaaaagcctgagtactgacctaagatggaattgtatgaactctgctctggagggaggggaggatgtccgtggaagttgtaagacttttatttttttgtgccatcaaatataggtaaaaataattgtgcaattctgctgtttaaacaggaactattggcctccttggccctaaatggaagggccgatattttaagttgattattttattgtaaattaatccaacctagttctttttaatttggttgaatgttttttcttgttaaatgatgtttaaaaaataaaaactggaagttcttggcttagtcataattctt

SEQ ID NO: 41 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 42 - DNA, 2949 bp, Homo sapiens
cgcctcccttccccctccccgcccgacagcggccgctcgggccccggctctcggttataagatggcggcgctgagcggtggcggtggtggcggcgcggagccgggccaggctctgttcaacggggacatggagcccgaggccggcgccggcgccggcgccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttggaattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtgaaccaaagaacacttgtgtggttaaagactagatataatttttccccaaactaaaatttatacttaacattggatttttaacatccaagggttaaaatacatagacattgctaaaaattggcagagcctcttctagaggctttactttctgttccgggtttgtatcattcacttggttattttaagtagtaaacttcagtttctcatgcaacttttgttgccagctatcacatgtccactagggactccagaagaagaccctacctatgcctgtgtttgcaggtgagaagttggcagtcggttagcctgggttagataaggcaaactgaacagatctaatttaggaagtcagtagaatttaataattctattattattcttaataatttttctataactatttctttttataacaatttggaaaatgtggatgtcttttatttccttgaagcaataaactaagtttctttttataaaaa

SEQ ID NO: 43 - protein, 766 aa, Homo sapiens [sequence omitted]

(19) United States
(12) Patent Application Publication
(10) Pub. No.: US 2016/0108479 A1
(43) Pub. Date: Apr. 21, 2016

(54) COMPOSITIONS AND METHODS FOR MULTIPLEX ANALYSIS OF NRAS AND BRAF NUCLEIC ACIDS

(71) Applicant: QIAGEN MANSFIELD, INC., Mansfield, MA (US)

(72) Inventors: Lilly I. KONG, Covina, CA (US); Kiran Madanahally DIVAKAR, Shrewsbury, MA (US)

(21) Appl. No.: 14/896,621

(22) PCT Filed: Jul. 30, 2014
(86) PCT No.: PCT/US2014/048807

§ 371 Date: Dec. 7, 2015

Related U.S. Application Data
(60) Provisional application No. 61/865,754, filed on Aug. 14, 2013.

(51) Int. Cl. C12Q 1/68

(52) U.S. Cl. CPC C12Q 1/6886; C12Q 2600/156; C12Q 2600/16

(57) ABSTRACT

Described herein are methods and assays relating to the detection of NRAS and/or BRAF alterations (e.g. variations in copy number and expression level, and/or the presence of mutations, including point mutations). Existing methods are limited in their clinical usefulness by, e.g., limited sensitivity, inter-lab discordance, or inability to provide the necessary multiplex ability. The methods and assays provided herein permit multimodal, multiplex assaying for faster, more cost-effective testing and screening of patients, permitting improved healthcare.

### CROSS-REFERENCE TO RELATED APPLICATIONS

This application claims benefit under 35 U.S.C. §119(e) of U.S. Provisional Application No. 61/865,754 filed Aug. 14, 2013, the content of which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jul. 29, 2014, is named 046264-078891-PCT_SL.txt and is 28,830 bytes in size.

### TECHNICAL FIELD

The technology described herein relates to assays and methods permitting the detection of the presence and/or absence of mutations, including point mutations.

### BACKGROUND

The development of personalized medicine has led to the identification of genes which, when mutated and/or altered, can contribute to disease. For example, the sequence encoding the gene can be mutated in a subject who has or is at risk of developing a given disease as compared to a wild-type or healthy subject.

For example, NRAS and BRAF are implicated in cancer and any given cancer cell can demonstrate mutations of one or both of NRAS and BRAF. These mutations have been linked to, e.g., disease severity and/or responsiveness to particular therapeutic options. Traditional approaches for detecting such mutations offer less multiplex ability than is necessary for comprehensive clinical diagnostics. The development of a multiplex assay can permit faster, more cost-effective testing and screening of patients, permitting improved healthcare.

### SUMMARY

The technology described herein is directed to methods and assays for detecting mutations of NRAS and/or BRAF, e.g. alterations in sequence. The inventors have developed assays and discovered methods for reliably determining the presence or absence of NRAS and/or BRAS mutations in a single multiplexed assay.

In one aspect, described herein is an assay for detecting mutations of NRAS and/or BRAF, the assay comprising contacting a portion of a nucleic acid sample with a set of primers, wherein the set of primers comprises subsets of primer pairs, wherein each primer pair amplifies a NRAS or BRAF sequence comprising a sequence variation; performing a PCR amplification regimen comprising cycles of strand separation, primer annealing, and primer extension on a reaction mixture comprising the portion of the sample and the one or more sets of primers; detecting the presence or absence of the amplicon for each primer pair; wherein the presence of an amplicon indicates the presence of the sequence variation for which that primer pair is specific; and wherein one or more of the primers are selected from the group consisting of SEQ ID NOs: 1-39.

In some embodiments, the primer pairs are selected from the group consisting of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39. In some embodiments, one or more sequence variations are point mutations. In some embodiments, the NRAS point mutation is selected from the group consisting of: G12D; G12S; G13A; G13C; G13D; G12R; G13V; Q61H1; Q61K; Q61L; Q61R1; and Q61R2. In some embodiments, the BRAF point mutation is selected from the group consisting of: V600D TG/AT; V600E T/A; V600E TG/AA; and V600K GT/AA. In some embodiments, the presence or absence of G12D; G12S; G13A; G13C; G13D; G12R; G13V; Q61H1; Q61K; Q61L; Q61R1; and Q61R2 is detected. In some embodiments, the presence or absence of V600D TG/AT; V600E T/A; V600E TG/AA; and V600K GT/AA is detected.

In some embodiments, the nucleic acid sample is prepared from a FFPE tumor sample. In some embodiments, the sample comprises tumor cells from a subject diagnosed with a condition selected from the group consisting of: gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer.

In some embodiments, one or more primers are dual domain primers. In some embodiments, the amplified products from two or more primer pairs of a primer set can be distinguished. In some embodiments, the amplified products from two or more primer pairs of a primer set are distinguished by being of distinct sizes. In some embodiments, the amplified products from two or more primer pairs of a primer set are distinguished by being labeled with different detectable labels. In some embodiments, the primers are present in the reaction mixture at about the concentrations of Example 1.

In one aspect, described herein is a composition comprising one or more primer pairs selected from the group consisting of molecules having the sequences of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39. In some embodiments, the composition can comprise primer pairs consisting of molecules having the sequences of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18. In some embodiments, the composition can comprise primer pairs consisting of molecules having the sequences of: SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39. In some embodiments, the primer pairs are specifically hybridized to target polynucleotides. In some embodiments, at least one member of each primer pair is fluorescently-labelled.

In one aspect, described herein is a composition comprising fluorescently-labelled amplification products resulting from the amplification of target polynucleotides with one or more primer pairs selected from the group consisting of molecules having the sequences of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

In some embodiments, the composition can further comprise reaction mixture components selected from the group consisting of: buffer; dNTPs; and DNA polymerase. In some embodiments, the composition can further comprise a nucleic acid sample prepared from a FFPE tumor sample. In some embodiments, the composition can further comprise tumor cells from a subject diagnosed with a condition selected from the group consisting of: gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and thyroid cancer. In some embodiments, the primers are present at about the concentrations of Example 1.

### BRIEF DESCRIPTION OF THE DRAWINGS

FIG. 1 depicts the results of multiplex detection of NRA/BRAF targets on ICEPlex system according to the embodiments described in Example 1. The upper panel demonstrates that 4 BRAF targets were detected in the TYE channel. The lower panel demonstrates the 12 NRAS targets were detected in the FAM channel.

FIG. 2 depicts the results of multiplex detection of reference gene controls as described in Example 1.

### DETAILED DESCRIPTION

Embodiments of the technology described herein are directed to methods and assays for detecting variations of NRAS and/or BRAF, e.g. variations in sequence (mutations), expression level, and/or gene copy number, and particularly multiplexed and multimodal assays and methods of detecting NRAS and/or BRAF variations. Intereference between primer sets and/or primer pair subsets is a problem encountered in multiplex PCT assays. The methods and compositions described herein improve upon existing technology at least by permitting multiplex detection of a plurality of different NRAS and/or BRAF mutations (e.g. SNPs) in a single reaction with high sensitivity and without significant cross-reaction between primer pairs.

A number of different NRAS and/BRAF variations are known, and distinguishing the presence and/or absence of such a group of variations in one sample is problematic with existing single reaction technologies because the variations are closely spaced, even overlapping in some cases. Background signals must be minimized or the assay will not reliably work to identify the specific mutations.

As used herein, the terms “NRAS” or “neuroblastoma RAS viral oncogene homolog” refer to a small GTPase Ras family protein encoded on chromosome 1. The sequences of NRAS are well known in the art for a number of species, eg human NRAS (NCBI Gene ID: 4893; SEQ ID NO: 40 (NCBI Ref Seq: NM_002524; mRNA); SEQ ID NO: 41 (NCBI Ref Seq: NP_002515; polypeptide)).

As used herein, the terms “BRAF” or “v-Raf murine sarcoma viral oncogene homolog B” refer to a Raf kinase family serine/threonine-specific protein kinase that interacts with AKT1; CRaf, HRAS, and YWHAB. The sequences of BRAF are well known in the art for a number of species, eg. human BRAF (NCBI Gene ID: 673; SEQ ID NO: 42 (NCBI Ref Seq: NM_004333; mRNA); SEQ ID NO: 43 (NCBI Ref Seq: NP_004324; polypeptide)).

In one aspect, described herein is an assay for detecting mutations of NRAS and/or BRAF, the assay comprising contacting a portion of a nucleic acid sample with a set of primers, wherein the set of primers comprises subsets of primer pairs, wherein each primer pair amplifies a NRAS or BRAF sequence comprising a sequence variation; performing a PCR amplification regimen comprising cycles of strand separation, primer annealing, and primer extension on a reaction mixture comprising the portion of the sample and the one or more sets of primers; detecting the presence or absence of the amplicon for each primer pair; wherein the presence of an amplicon indicates the presence of the sequence variation for which that primer pair is specific.

In some embodiments, one or more of the primers are selected from the group consisting of molecules with the sequences of SEQ ID NOs: 1-39. In some embodiments, one or more of the primers are selected from the group consisting of molecules with the sequences of SEQ ID NOs: 1-18. In some embodiments, one or more of the primers are selected from the group consisting of molecules with the sequences of SEQ ID NOs: 19-39. In some embodiments, the primers consist of the molecules with the sequences of SEQ ID NOs: 1-18. In some embodiments, the primers consist of the molecules with the sequences of SEQ ID NOs: 19-39.

Variations of BRAF and/or NRAS, can be of use in diagnosis, prognosis, and/or selection of treatment. In some embodiments, the multiple mutations of BRAF and/or NRAS are detected in the same reaction mixture, e.g. in the same tube, well, or vessel. In some embodiments, the assays described herein occur in a single tube, e.g multiple subsets of primer pairs are present in a single reaction mixture and/or vessel or container. Thus, in said embodiments, a single amplification regimen will provide data regarding the presence and/or absence of multiple mutations.

In one aspect, the assays and methods described herein relate to detecting the presence of sequence variations in NRAS and/or BRAF. As used herein, “sequence variations” can refer to substitutions, insertions, deletions, duplications, or rearrangements. In some embodiments, one or more sequence variations are point mutations.

A sequence variation, including, e.g. a point mutation, e.g. a single nucleotide polymorphism (SNP), can be phenotypically neutral or can have an associated variant phenotype that distinguishes it from that exhibited by the predominant sequence at that locus. As used herein, “neutral polymorphism” refers to a polymorphism in which the sequence variation does not alter gene function, and “mutation” or “functional polymorphism” refers to a sequence variation which does alter gene function, and which thus has an associated phenotype. Sequence variations of a locus occurring in a population are referred to as alleles. When referring to the genotype of an individual with regard to a specific locus at which two or more alleles occur within a population, the “predominant allele” is that which occurs most frequently in the population in question (i.e., when there are two alleles, the allele that occurs in greater than 50% of the population is the predominant allele; when there are more than two alleles, the “predominant allele” is that which occurs in the subject population at the highest frequency, e.g., at least 5% higher frequency, relative to the other alleles at that site). The term “variant allele” is used to refer to the allele or alleles occurring less frequently than the predominant allele in that population (e.g., when there are two alleles, the variant allele is that which occurs in less than 50% of the subject population; when there are more than two alleles, the variant alleles are all of those that occur less frequently, e.g., at least 5% less frequently, than the predominant allele). Sequence variations can be present in (and therefore, detected in) the gDNA and/or mRNA of a gene.

In some embodiments, the sequence variant can be a point mutation. As used herein, a “point mutation” refers to the identity of the nucleotide present at a site of a mutation in the mutant copy of a genomic locus (including insertions and deletions), i.e. it refers to an alteration in the sequence of a nucleotide at a single base position from the wild type sequence. A SNP (single nucleotide polymorphism) is one type of point mutation that occurs at the same genomic locus between different individuals in a population. Point mutations may be somatic in that they occur between different cells in the same individual.

In some embodiments, the sequence variation can be a single nucleotide polymorphism (SNP). As used herein, a “single nucleotide polymorphism” or “SNP” refers to nucleic acid sequence variation at a single nucleotide residue, including a single nucleotide deletion, insertion, or base change or substitution. SNPs can be allelic. Some SNPs have defined phenotypes, e.g. disease phenotypes, while others have no known associated phenotype. SNP detection methods described herein can be used for the prediction of phenotypic characteristics, e.g. prediction of responsiveness or sensitivity to drugs. In this regard, SNP genotyping as described herein and known in the art is not necessarily diagnostic of disease or susceptibility to disease.

As noted, in some embodiments, an alteration comprises a SNP. At least four alleles of a SNP locus are possible, although SNPs that vary only between two nucleotides at the target site are not uncommon. In some embodiments, the methods and compositions described herein relate to a subset of primer pairs that can detect a single allele of a SNP locus. In some embodiments, the methods and compositions described herein relate to a set of primers that can detect two alleles of a SNP locus (i.e. the methods and compositions can relate to an assay that permits the affirmative detection of two SNP alleles, or “biphasic” genotyping of that SNP). In some embodiments, the methods and compositions described herein relate to a set of primers that can detect three alleles of a SNP locus (i.e. the methods and compositions can relate to an assay that permits the affirmative detection of three SNP alleles, or “triphasic” genotyping of that SNP). In some embodiments, the methods and compositions described herein relate to an assay that permits affirmative detection of four alleles of a SNP locus (i.e. the methods and compositions can relate to a multiplex detection of four SNP alleles, or “quaduphasic” genotyping of that SNP). In some embodiments, the predominant and/or wild-type allele of a SNP is detected. In some embodiments, the predominant and/or wild-type allele of a SNP is not detected. By “affirmatively detected” is meant that the assay permits the amplification of that specific allele. An alternative to affirmative detection can be used, for example, when there are only two possibilities known to exist at the SNP site. In this instance, the assay can be designed such that one of the two variants is amplified, and the other is not; the assay can affirmatively detect that amplified variant and passively detect the other, i.e. the lack of a product means the other allele or variant is present.

In some embodiments, the NRAS and/or BRAF sequence variation(s) can be point mutations. In some embodiments, a NRAS point mutation can be a point mutation resulting in one of the following amino acid residue changes: G12D; G12S; G13A; G13C; G13D; G12R; G13V; Q61H1; Q61K; Q61L; Q61R1; and Q61R2. In some embodiments, a BRAF point mutation can be a point mutation resulting in one of the following amino acid residue changes: V600D TG/AT; V600E T/A; V600E TG/AA; and V600K GT/AA.

In various embodiments, the methods and compositions described herein relate to performing a PCR amplification regimen with at least one set of oligonucleotide primers. As used herein, “primer” refers to a DNA or RNA polynucleotide molecule or an analog thereof capable of sequence-specifically annealing to a polynucleotide template and providing a 3′ end that serves as a substrate for a template-dependent polymerase to produce an extension product which is complementary to the polynucleotide template. The conditions for initiation and extension usually include the presence of at least one, but more preferably all four different deoxyribonucleoside triphosphates and a polymerization-inducing agent such as DNA polymerase or reverse transcriptase, in a suitable buffer (in this context “buffer” includes solvents (generally aqueous) plus necessary cofactors and reagents which affect pH, ionic strength, etc.) and at a suitable temperature. A primer useful in the methods described herein is generally single-stranded, and a primer and its complement can anneal to form a double-stranded polynucleotide. Primers according to the methods and compositions described herein can be less than or equal to 300 nucleotides in length, e.g., less than or equal to 300, or 250, or 200, or 150, or 100, or 90, or 80, or 70, or 60, or 50, or 40, and preferably 30 or fewer, or 20 or fewer, or 15 or fewer, but at least 10 nucleotides in length.

As used herein, the term “set” means a group of nucleic acid samples, primers or other entities. A set will comprise a known number of, and at least two of such entities. A set of primers comprises at least one forward primer and at least one reverse primer specific for a target sequence. A set of primers will comprise at least one primer pair subset, e.g. one primer pair subset, two primer pair subsets, three primer pair subsets, four primer pair subsets, five primer pair subsets, six primer pair subsets, or more primer pair subsets. In some embodiments, a set of primers can comprise primer pair subsets that detect mutations in NRAS, BRAF, or both NRAS and BRAF.

Thus, as used herein, “a primer pair subset” refers to a group of at least two primers, including a forward primer and a reverse primer, one of which anneals to a first strand of a target nucleic acid sequence and the other of which anneals to a complement of the first strand. In some embodiments, the first primer of a primer pair subset can anneal to a first strand of a target nucleic acid sequence and the second primer of a primer pair subset (e.g., reverse primer), can anneal to the complement of that strand. The orientation of the primers when annealed to the target and/or its complement can be such that nucleic acid synthesis proceeding from primer extension of a one primer of the primer pair subset would produce a nucleic acid sequence that is complementary to at least one region of the second primer of the primer pair subset. The “first strand” of a nucleic acid target and/or sequence can be either strand of a double-stranded nucleic acid comprising the sequence of the target nucleotide and/or target site locus, but once chosen, defines its complement as the second strand. Thus, as used herein, a “forward primer” is a primer which anneals to a first strand of a nucleic acid target, while a “reverse primer” of the same set is a primer which anneals to the complement of the first strand of the nucleic acid target.

As used herein, “specific” when used in the context of a primer specific for a target nucleic acid refers to a level of complementarity between the primer and the target such that there exists an annealing temperature at which the primer will anneal to and mediate amplification of the target nucleic acid and will not anneal to or mediate amplification of non-target sequences present in a sample. In the context of primer pair subsets that amplify sequence variations, at least one of the primers of the subset is specific for the sequence variation, e.g. the primer pair subset will not amplify the wild-type sequence not comprising the sequence variation.

Methods of making primers are well known in the art, and numerous commercial sources offer oligonucleotide synthesis services suitable for providing primers according to the methods and compositions described herein, e.g. INVITROGEN™ Custom DNA Oligos; Life Technologies; Grand Island, N.Y. or custom DNA Oligos from IDT; Coralville, Iowa).

In some embodiments, one or more primers can be dual domain primers. Dual domain primers are described in detail in PCT/US13/27383, filed Feb. 22, 2013 and published as WO2013/126743; the contents of which are incorporated by reference herein in its entirety. Dual domain primers can permit increased sensitivity, e.g. by reducing background from various sources.

Exemplary embodiments of primers are described herein. In some embodiments, one or more primers can be selected from the group consisting of SEQ ID NOs: 1-39. Exemplary subsets of primer pairs are depicted in Tables 1 and 4. In some embodiments, the primers can be present in the reaction mixture(s) at about the concentrations described in Example 1.

In some embodiments, the primer pairs can be selected from the group consisting of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

In some embodiments, the primer pairs can be selected from the group consisting of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; and SEQ ID NO:s 17 and 18. In some embodiments, the primer pairs comprise the group consisting of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; and SEQ ID NO:s 17 and 18. In some embodiments, the primer pairs consist of: SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; and SEQ ID NO:s 17 and 18.

In some embodiments, the primer pairs can be selected from the group consisting of: SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39. In some embodiments, the primer pairs can be selected from the group consisting of: SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; and SEQ ID NO:s 31 and 32. In some embodiments, the primer pairs comprise the group consisting of: SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; and SEQ ID NO:s 31 and 32. In some embodiments, the primer pairs consist of: SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; and SEQ ID NO:s 31 and 32.

The methods and compositions described herein relate to performing a polymerase chain reaction (PCR) amplification regimen. As used herein, the term “amplification regimen” refers to a process of specifically amplifying, i.e., increasing the abundance of, a nucleic acid sequence of interest, and more particularly, the exponential amplification occurring when the products of a previous polymerase extension serve as templates for the successive rounds of extension. A PCR amplification regimen according to the invention comprises at least two, and preferably at least 5, 10, 15, 20, 25, 30, 35 or more iterative cycles, where each cycle comprises the steps of: 1) strand separation (e.g., thermal denaturation); 2) oligonucleotide primer annealing to template molecules; and 3) nucleic acid polymerase extension of the annealed primers. Conditions and times necessary for each of these steps can be devised by one of ordinary skill in the art. An amplification regimen according to the methods described herein is preferably performed in a thermal cycler, many of which are commercially available.

In some embodiments, the nucleic acid sample can be subjected to reverse transcription prior to the PCR amplification regimen described herein, e.g. when the an alternation in an mRNA is to be determined as described herein. Reverse transcription protocols and reagents are well known in the art and are commercially available. An exemplary embodiment of a reverse transcription regimen is as follows: 5 uL of a nucleic acid sample comprising both RNA and gDNA (e.g. 25 ng of RNA and 2.5 ng of gDNA) are added to a reaction mixture comprising RT buffer, 0.5 mM dNTPs, 5 nM RT primers, and 20 units of SuperScript III™ reverse transcriptase (RNA-dependent DNA polymerase). The reaction is then incubated at 50° C. for 30 minutes, 90° C. for 5 minutes, and 4° C. for 5 minutes.

PCR requires the use of a nucleic acid polymerase. As used herein, the phrase “nucleic acid polymerase” refers an enzyme that catalyzes the template-dependent polymerization of nucleoside triphosphates to form primer extension products that are complementary to the template nucleic acid sequence. A nucleic acid polymerase enzyme initiates synthesis at the 3′ end of an annealed primer and proceeds in the direction toward the 5′ end of the template. Numerous nucleic acid polymerases are known in the art and commercially available. One group of preferred nucleic acid polymerases are thermostable, i.e., they retain function after being subjected to temperatures sufficient to denature annealed strands of complementary nucleic acids, e.g. 94° C., or sometimes higher. In some embodiments, the polymerase can be delta-exo-Apta Taq Polymerase.

As understood in the art, PCR requires cycles including a strand separation step generally involving heating of the reaction mixture. As used herein, the term “strand separation” or “separating the strands” means treatment of a nucleic acid sample such that complementary double-stranded molecules are separated into two single strands available for annealing to an oligonucleotide primer. More specifically, strand separation according to the methods described herein is achieved by heating the nucleic acid sample above its Tₘ. Generally, for a sample containing nucleic acid molecules in buffer suitable for a nucleic acid polymerase, heating to 94° C. is sufficient to achieve strand separation. An exemplary buffer contains 50 mM KCl, 10 mM Tric-HCl (pH 8.8@25° C.), 0.5 to 3 mM MgCl₂, and 0.1% BSA.

As also understood in the art, PCR requires annealing primers to template nucleic acids. As used herein, “anneal” refers to permitting two complementary or substantially complementary nucleic acids strands to hybridize, and more particularly, when used in the context of PCR, to hybridize such that a primer extension substrate for a template-dependent polymerase enzyme is formed. Conditions for primer-target nucleic acid annealing vary with the length and sequence of the primer and are based upon the calculated Tₘ for the primer. Generally, an annealing step in an amplification regimen involves reducing the temperature following the strand separation step to a temperature based on the calculated Tₘ for the primer sequence, for a time sufficient to permit such annealing.

Tₘ can be readily predicted by one of skill in the art using any of a number of widely available algorithms (e.g., OLIGO™ (Molecular Biology Insights Inc. Colorado) primer design software and VENTRO NTI™ (Invitrogen, Inc. California) primer design software and programs available on the internet, including Primer3 and Oligo Calculator). For example, Tₘ's can be calculated using the NetPrimer software (Premier Biosoft; Palo Alto, Calif.; and freely available on the world wide web at http://www.premierbiosoft.com/netprimer/netprlaunch/Help/xnetprlaunch.html). The Tₘ of a primer can also be calculated using the following formula, which is used by NetPrimer software and is described in more detail in Frieir et al. PNAS 1986 83:9373-9377 which is incorporated by reference herein in its entirety.

Tₘ=ΔH/(ΔS+R*ln(C/4))+16.6 log([K⁺]/(1+0.7[K⁺]))−273.15

wherein, ΔH is enthalpy for helix formation; ΔS is entropy for helix formation; R is molar gas constant (1.987 cal/° C.*mol); C is the nucleic acid concentration; and [K⁺] is salt concentration. For most amplification regimens, the annealing temperature is selected to be about 5° C. below the predicted Tₘ, although temperatures closer to and above the Tₘ (e.g., between 1° C. and 5° C. below the predicted Tₘ or between 1° C. and 5° C. above the predicted Tₘ) can be used, as can, for example, temperatures more than 5° C. below the predicted Tₘ (e.g., 6° C. below, 8° C. below, 10° C. below or lower). Generally, the closer the annealing temperature is to the Tₘ, the more specific is the annealing. The time allowed for primer annealing during a PCR amplification regimen depends largely upon the volume of the reaction, with larger volumes requiring longer times, but also depends upon primer and template concentrations, with higher relative concentrations of primer to template requiring less time than lower relative concentrations. Depending upon volume and relative primer/template concentration, primer annealing steps in an amplification regimen can be on the order of 1 second to 5 minutes, but will generally be between 10 seconds and 2 minutes, preferably on the order of 30 seconds to 2 minutes.

As used herein, “substantially anneal” refers to a degree of annealing during a PCR amplification regimen which is sufficient to produce a detectable level of a specifically amplified product.

PCR also relies upon polymerase extension of annealed primers at each cycle. As used herein, the term “polymerase extension” means the template-dependent incorporation of at least one complementary nucleotide, by a nucleic acid polymerase, onto the 3′ end of an annealed primer. Polymerase extension preferably adds more than one nucleotide, preferably up to and including nucleotides corresponding to the full length of the template. Conditions for polymerase extension vary with the identity of the polymerase. The temperature used for polymerase extension is generally based upon the known activity properties of the enzyme. Although, where annealing temperatures are required to be, for example, below the optimal temperatures for the enzyme, it will often be acceptable to use a lower extension temperature. In general, although the enzymes retain at least partial activity below their optimal extension temperatures, polymerase extension by the most commonly used thermostable polymerases (e.g., Taq polymerase and variants thereof) is performed at 65° C. to 75° C., preferably about 68-72° C.

Primer extension is performed under conditions that permit the extension of annealed oligonucleotide primers. As used herein, the term “conditions that permit the extension of an annealed oligonucleotide such that extension products are generated” refers to the set of conditions including, for example temperature, salt and co-factor concentrations, pH, and enzyme concentration under which a nucleic acid polymerase catalyzes primer extension. Such conditions will vary with the identity of the nucleic acid polymerase being used, but the conditions for a large number of useful polymerase enzymes are well known to those skilled in the art. One exemplary set of conditions is 50 mM KCl, 10 mM Tric-HCl (pH 8.8@25° C.), 0.5 to 3 mM MgCl₂, 200 uM each dNTP, and 0.1% BSA at 72° C., under which Taq polymerase catalyzes primer extension.

In some embodiments, the thermocycling conditions can be in accordance with the protocol depicted in Example 1.

In some embodiments, a buffer for use in the methods and assays described herein can comprise Tris buffer, trehalose, potassium acetate, glycerol, betaine, magnesium chloride, potassium chloride, ammonium sulphate, DMSO, DTT, BSA, dNTPs, Tween-20 and polymerase. In some embodiments, a buffer for use in the methods and assays described herein can comprise 10-400 mM Tris buffer (pH 7.5 to 9.5), 2-20% trehalose, 10-300 mM potassium acetate, 1-7.5% glycerol, 100 mM to 2M betaine, 2.5-12.5 mM magnesium chloride, 1-10 mM potassium chloride, 1-10 mM ammonium sulphate, 0.1-2% DMSO, 1-10 mM DTT, 10-1,000 ug/mL BSA, 50-400 mM dNTP, 0-1% Tween-20 and 1-10 enzyme units of polymerase.

As used herein, “amplified product” or “amplicon” refers to polynucleotides resulting from a PCR reaction that are copies of a portion of a particular target nucleic acid sequence and/or its complementary sequence, which correspond in nucleotide sequence to the template nucleic acid sequence and/or its complementary sequence. An amplified product, as described herein will generally be double-stranded DNA, although reference can be made to individual strands thereof.

The methods described herein use PCR to quantitate or evaluate gene mutation. For any of the methods described herein, quantitation can be achieved by withdrawing samples from the PCR reaction at plural cycles and separating and detecting the amounts of the amplicons in the sample withdrawn. The amplification profile for each amplicon measured in this manner permits the quantitation of initial template. See, e.g., U.S. Pat. No. 8,321,140 and U.S. Patent Application No. 2013/0053274; which are incorporated by reference herein in their entireties.

In some embodiments, the methods and compositions described herein relate to multiplex PCR. As used herein, “multiplex PCR” refers to a variant of PCR where simultaneous amplification of more than one target nucleic acid sequence in one reaction vessel and subsequent or concurrent detection of the multiple products can be accomplished by using more than one pair of primers in a set (e.g., at least more than one forward and/or more than one reverse primer). Multiplex amplification can be useful not only for detecting the presence of a plurality of targets but also for the analysis, detection, and/or genotyping of deletions, mutations, and polymorphisms, and/or expression level and/or for quantitative assays. Multiplex can refer to the detection of between 2-1,000 different target sequences and/or alterations of a target nucleic acid in a single reaction. As used herein, multiplex refers to the detection of any range between 2-1,000, e.g., between 5-500, 25-1000, or 10-100 different target sequences in a single reaction, etc. By way of non-limiting example, a multiplex PCR reaction as part of a method described herein can affirmatively detect the presence of two or more possible alleles of at least two SNPs at at least two different allelic target site loci in a single reaction. The term “multiplex” as applied to PCR implies that there are primers specific for at least two different target sequences in the same PCR reaction. Thus, a reaction in which there are primer sets specific for two different target sequences is considered a multiplex amplification even if only one (or even none) of the at least two target sequences is actually detected in a given sample. Thus, in some embodiments, multiplex PCR can also refer to a reaction containing multiple pairs of primers, wherein the reaction can result in one or multiple specific amplified products when one or multiple targets are present in the reaction.

Quantitative aspects can be facilitated, for example, by repeated sampling at any time during or after an amplification reaction, followed by separation and detection of the amplification products. Sampling can, for example, comprise removing an aliquot of the reaction. Sampling can occur, for example, at the end of every cycle, or at the end of every several cycles, e.g. every two cycles, every three cycles, every four cycles etc. While a uniform sample interval will most often be desired, there is no requirement that sampling be performed at uniform intervals. As just one example, the sampling routine can involve sampling after every cycle for the first five cycles, and then sampling after every other cycle or vice versa.

Sampling or dispensing of an aliquot from an amplification reaction can be performed in any of several different general formats. The sampling or removal method can depend on any of a number of factors including, but not limited to, the equipment available, the number of samples to be analyzed, and the timing of detection relative to sample collection (e.g., concurrently vs. sequential). The exact method of removal or extrusion of samples is not necessarily a limitation of the methods described herein. Sampling is preferably performed with an automated device, especially for high throughput applications. Sampling can also be performed using direct electrokinetic or hydrodynamic injection from a PCR reaction into a capillary electrophoretic device. The method of sampling used in the methods is preferably adapted to minimize contamination of the cycling reaction(s), by, for example, using pipetting tips or needles that are either disposed of after a single aliquot is withdrawn, or by using the same tip or needle for dispensing the sample from the same PCR reaction vessel. Methods for simultaneous sampling and detection are known to those skilled in the art (see, e.g., US Patent Application Publication 2004/0166513, incorporated herein by reference).

The amount of nucleic acid and/or volume of an aliquot dispensed at the sampling step can vary, depending, for example, upon the total volume of the amplification reaction, the sensitivity of product detection, and the type of sampling and/or separation used. Amplification volumes can vary from several microliters to several hundred microliters (e.g., 5 μl, 10 μl, 20 μl, 40 μl, 60 μl, 80 μl, 100 μl, 120 μl, 150 μl, or 200 μl or more), preferably in the range of 10-150 μl, more preferably in the range of 10-100 μl. The exact volume of the amplification reaction is not a limitation of the invention. Aliquot volumes can vary from 0.01% to 30% of the reaction mixture. Electrokinetic injection into capillary electrophoresis capillaries will generally load nucleic acid but not appreciably diminish the volume of the sampled reaction. The amplification regimen can be performed on plural independent nucleic acid amplification mixtures, optionally in a multiwell container. The container(s) in which the amplification reaction(s) are preformed is not necessarily a limitation of the methods described herein.

In various embodiments, the methods and compositions described herein relate to detecting amplified products (e.g. amplicons) for each target nucleic acid sequence, e.g. for each target alteration. In some embodiments, the detecting of the amplified product for each target nucleic acid sequence affirmatively indicates the presence of the target nucleic acid sequence in a sample. In some embodiments, the quantitative detecting of the amplified product for each target nucleic acid sequence indicates the level of that target nucleic acid sequence in a sample.

In some embodiments, the methods and compositions described herein relate to the amplified products of two or more primer pair subsets which should be distinguishable from each other. In some embodiments, the methods and compositions described herein relate to PCR amplification regimens wherein the amplified products of two or more primer pair subsets can be distinguished by being of distinct sizes. As used herein, a nucleic acid is of a “distinct size” if it is resolvable from nucleic acids of a different size. “Different sizes” refers to nucleic acid molecules that differ by at least one nucleotide in length. Generally, distinctly sized amplification products useful according to the methods described herein differ by a number of nucleotides greater than or equal to the limit of resolution for the separation process used in a given separation or detection method. For example, when the limit of resolution of separation is one base, distinctly sized amplification products differ by at least one base in length, but can differ by 2 bases, 5 bases, 10 bases, 20 bases, 50 bases, 100 bases or more. When the limit of resolution is, for example, 10 bases, distinctly sized amplification products will differ by at least 10 bases, but can differ by 11 bases, 15 bases, 20 bases, 30 bases, 50 bases, 100 bases or more.

In some embodiments, both the lengths of the primers or any portion thereof and the lengths of the segment of the target nucleic acid sequence that they anneal to can vary. Variation in the length of target sequence amplified, e.g. by chosen placement of the forward and reverse primers further or closer apart, is a straightforward approach to ensuring ready distinctions between products from different targets. Variation in the length of the primer, especially the 5′ tail regions of dual domain primers, is particularly effective, e.g. distinguishing the products of specific alleles of a given target locus in an assay.

In some embodiments the amplified products are distinguished by being labeled with different detectable labels. In some embodiments, the label is incorporated into a primer. In some embodiments, the label is conjugated to a primer.

In some embodiments, the label is bound to the primer after the PCR amplification regimen is complete. In some embodiments, the label is conjugated to an oligonucleotide or antibody or portion thereof that specifically binds to primer, or to a moiety attached thereto.

Two detectable labels are considered different if the signal from one label can be distinguished from the signal from the other. Detectable labels can comprise, for example, a light-absorbing dye, a fluorescent dye, or a radioactive label. Fluorescent dyes are preferred. Generally, a fluorescent signal is distinguishable from another fluorescent signal if the peak emission wavelengths are separated by at least 20 nm Greater peak separation is preferred, especially where the emission peaks of fluorophores in a given reaction are wide, as opposed to narrow or more abrupt peaks.

Detectable labels, methods of detecting them, and methods of incorporating them into or coupling and/or binding them to an amplified product are well known in the art. The following is provided by way of non-limiting example.

In some embodiments, detectable labels can include labels that can be detected by spectroscopic, photochemical, biochemical, immunochemical, electromagnetic, radiochemical, or chemical means, such as fluorescence, chemifluorescence, or chemiluminescence, or any other appropriate means.

The detectable labels used in the methods described herein can be primary labels (where the label comprises a moiety that is directly detectable or that produces a directly detectable moiety) or secondary labels (where the detectable label binds to another moiety to produce a detectable signal, e.g., as is common in immunological labeling using secondary and tertiary antibodies).

The detectable label can be linked by covalent or non-covalent means to nucleic acids. Alternatively, a detectable label can be linked such as by directly labeling a molecule that achieves binding to another nucleic acid via a ligand-receptor binding pair arrangement or other such specific recognition molecules. Detectable labels can include, but are not limited to radioisotopes, bioluminescent compounds, chromophores, antibodies, chemiluminescent compounds, fluorescent compounds, metal chelates, and enzymes.

In some embodiments, a detectable label can be a fluorescent dye molecule, or fluorophore including, but not limited to fluorescein, phycoerythrin, Cy3™, Cy5™, allophycocyanine, Texas Red, peridenin chlorophyll, cyanine, tandem conjugates such as phycoerythrin-Cy5™, green fluorescent protein, rhodamine, fluorescein isothiocyanate (FITC) and Oregon Green™, rhodamine and derivatives (e.g., Texas red and tetrarhodimine isothiocynate (TRITC)), biotin, phycoerythrin, AMCA, CyDyes™, 6-carboxythiorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescein (JOE or J), N,N,N′,N′-tetramethyl-6carboxyrhodamine (TAMRA or T), 6-carboxy-X-rhodamine (ROX or R), 5-carboxyrhodamine-6G (R6G5 or G5), 6-carboxyrhodamine-6G (R6G6 or G6), and rhodamine 110; cyanine dyes, e.g. Cy3, Cy5 and Cy7 dyes; coumarins, e.g umbelliferone; benzimide dyes, e.g. Hoechst 33258; phenanthridine dyes, e.g. Texas Red; ethidium dyes; acridine dyes; carbazole dyes; phenoxazine dyes; porphyrin dyes; polymethine dyes, e.g. cyanine dyes such as Cy3, Cy5, etc; BODIPY dyes and quinoline dyes.

In some embodiments, a detectable label can be a radiolabel including, but not limited to ³H, ¹²⁵I, ³⁵S, ¹⁴C, ³²P, and ³³P.

In some embodiments, a detectable label can be an enzyme including, but not limited to horseradish peroxidase and alkaline phosphatase. An enzymatic label can produce, for example, a chemiluminescent signal, a color signal, or a fluorescent signal.

In some embodiments, a detectable label is a chemiluminescent label, including, but not limited to luminol, luciferin or lucigenin.

In some embodiments, a detectable label can be a spectral colorimetric label including, but not limited to colloidal gold or colored glass or plastic (e.g., polystyrene, polypropylene, and latex) beads.

In some embodiments, the methods and compositions described herein relate to PCR amplification regimens wherein the amplified products of two or more primer pair subsets can be distinguished by being sequenced. Methods of sequencing nucleic acids are well known in the art and commercial sequencing services are widely available (e.g. Genscript; Piscataway, N.J.).

In some embodiments, the methods and compositions described herein relate to PCR amplification regimens wherein the amplified products of two or more primer pair subsets can be distinguished by melting-curve analysis. Methods of melting-curve analyses are well known in the art (e.g. Ririe et al. Analytical Biochemistry 1997 245:154-160; Wittwer et al. Clinical Chemistry 2003 49:853-860; and Liew et al. Clinical Chemistry 2007 50:1156-1164; which are incorporated by reference herein in their entireties).

Direct detection of size-separated amplification products is preferred. However, in some embodiments, the methods and compositions described herein relate to PCR amplification regimens wherein the amplified products of two or more primer pair subsets can be distinguished by oligonucleotide hybridization. One having ordinary skill in the art, using the sequence information of the target nucleic acid sequences, can design probes which are fully complementary to a single target and not to other target nucleic acid sequences. Hybridization conditions can be routinely optimized to minimize background signal by non-fully complementary hybridization. Hybridization probes can be designed to hybridize to the primer sequence, or part of the amplified product not comprised by the primer, provided that the sequence to which the probe will hybridize distinguishes it from at least one other amplified product present in the reaction.

In some embodiments, the PCR amplification regimen described herein is a multiplex regimen. In some embodiments, an amplification product of one primer pair subset can be distinguished from the amplification products of other primer pair subsets by at least two approaches, e.g. each primer subset can produce amplicons with a unique detectable fluorescence label and a detectable size difference. In some embodiments, an amplification product of one primer pair subset can be distinguished from the amplification products of other primer pair subsets by at least two approaches, e.g. 1) each primer subset specific for an NRAS mutation can produce amplicons with a first detectable fluorescence label while each primer subset specific for a BRAF mutation can produce amplicons with a second detectable fluorescence label and 2) each primer subset can produce an amplicon having a detectable size difference. In some embodiments, an amplification product of one primer pair subset can be distinguished from the amplification products of other primer pair subsets by at least two approaches, e.g. 1) each primer subset specific for an NRAS mutation can produce amplicons with a first detectable fluorescence label while each primer subset specific for a BRAF mutation can produce amplicons with a second detectable fluorescence label and 2) each primer subset having the same detectable fluorescence label can produce an amplicon having a detectable size difference (e.g. all amplicons produced from BRAF target sequences can have the same fluorescent label and be differentiated from each other by size while they are differentiated from the amplicons produced by NRAS target sequences by having a different fluorescence label).

In some embodiments, the primer sets described herein can have a cross-hybridization rate of less than about 30%, e.g. under the PCR amplification conditions. In some embodiments, the primer sets described herein can have a cross-hybridization rate of less than about 20%, e.g. under the PCR amplification conditions. In some embodiments, the primer sets described herein can have a cross-hybridization rate of less than about 10%, e.g. under the PCR amplification conditions.

The methods and compositions described herein relate to the detection of the presence and/or level of a target nucleic acid sequence, e.g. the presence and/or level of a gene alteration in a sample. A target nucleic acid can be an RNA or a DNA. A target nucleic acid can be a double-stranded (ds) nucleic acid or a single-stranded (ss) nucleic acid, e.g. a dsRNA, a ssRNA, a dsDNA, or a ssDNA. Non-limiting examples of target nucleic acids include a nucleic acid sequence, a nucleic acid sequence comprising a mutation, a nucleic acid sequence comprising a deletion, a nucleic acid sequence comprising an insertion, a sequence variant, an allele, a polymorphism, a point mutation, a SNP, a microRNA, a protein coding RNA, a non-protein coding RNA, an mRNA, a nucleic acid from a pathogen (e.g. a bacterium, a virus, or a parasite), a nucleic acid associated with a disease or a likelihood of having or developing a disease (e.g. a marker gene, a polymorphism associated with a disease or a likelihood of having or developing a disease, or an RNA, the expression of which is associated with a disease or a likelihood of having or developing a disease).

A sample useful herein will comprise nucleic acids. In some embodiments, a sample can further comprise proteins, cells, fluids, biological fluids, preservatives, and/or other substances. In some embodiments, a sample can be obtained from a subject. In some embodiments, a sample can be a biological sample obtained from the subject. In some embodiments a sample can be a diagnostic sample obtained from a subject. By way of non-limiting example, a sample can be a cheek swab, blood, serum, plasma, sputum, cerebrospinal fluid, urine, tears, alveolar isolates, pleural fluid, pericardial fluid, cyst fluid, tumor tissue, tissue, a biopsy, saliva, an aspirate, or combinations thereof. In some embodiments, a sample can be obtained by resection or biopsy.

In some embodiments, the sample is a clarified fluid sample, for example, by centrifugation. In some embodiments, the sample is clarified by low-speed centrifugation (e.g. 3,000×g or less) and collection of the supernatant comprising the clarified fluid sample.

In some embodiments, the sample can be freshly collected. In some embodiments, the sample can be stored prior to being used in the methods and compositions described herein. In some embodiments, the sample is an untreated sample. As used herein, “untreated sample” refers to a biological sample that has not had any prior sample pre-treatment except for dilution and/or suspension in a solution.

In some embodiments, a sample can be obtained from a subject and preserved or processed prior to being utilized in the methods and compositions described herein. By way of non-limiting example, a sample can be embedded in paraffin wax, refrigerated, or frozen. A frozen sample can be thawed before determining the presence of a nucleic acid according to the methods and compositions described herein. In some embodiments, the sample can be a processed or treated sample. Exemplary methods for treating or processing a sample include, but are not limited to, centrifugation, filtration, sonication, homogenization, heating, freezing and thawing, contacting with a preservative (e.g. anti-coagulant or nuclease inhibitor) and any combination thereof. In some embodiments, the sample can be treated with a chemical and/or biological reagent. Chemical and/or biological reagents can be employed to protect and/or maintain the stability of the sample or nucleic acid comprised by the sample during processing and/or storage. In addition, or alternatively, chemical and/or biological reagents can be employed to release nucleic acids from other components of the sample. By way of non-limiting example, a blood sample can be treated with an anti-coagulant prior to being utilized in the methods and compositions described herein. The skilled artisan is well aware of methods and processes for processing, preservation, or treatment of samples for nucleic acid analysis.

In some embodiments, the nucleic acid sample can be prepared from a FFPE tumor sample. In some embodiments, the sample can comprise tumor cells from a subject having, or diagnosed as having gastric cancer; renal cancer; cholanigoma; lung cancer; brain cancer; cervical cancer; colon cancer; head and neck cancer; hepatoma; non-small cell lung cancer; melanoma; mesothelioma; multiple myeloma; ovarian cancer; sarcoma; and/or thyroid cancer. See, e.g. Sattler et al. Ther Adv Med Oncol 2011 3:171-184; which is incorporated by reference herein in its entirety. “Expander” polynucleotides can be incorporated and/or added to the methods and compositions described herein to improve performance with, e.g., FFPE samples. Expander polynucleotides and methods of using the same are described in International Patent Publication WO 2013/010074.

In some embodiments, the nucleic acid present in a sample is isolated, enriched, or purified prior to being utilized in the methods and compositions described herein. Methods of isolating, enriching, or purifying nucleic acids from a sample are well known to one of ordinary skill in the art. By way of non-limiting example, kits for isolation of genomic DNA from various sample types are commercially available (e.g. Catalog Nos. 51104, 51304, 56504, and 56404; Qiagen; Germantown, Md.).

The terms “subject” and “individual” are used interchangeably herein, and refer to an organism from which a sample is obtained. A subject can be any organism for which it is desired to determine the presence of a nucleic acid in the organism or one or more cells comprising or contained within that organism. As used herein, a “subject” can mean an organism, e.g. a bacterium, a parasite, a plant, or an animal. In some embodiments, a subject can be a human or animal. Usually the animal is a vertebrate such as a primate, rodent, domestic animal or game animal. Primates include chimpanzees, cynomologous monkeys, spider monkeys, and macaques, e.g., Rhesus monkeys. Rodents include, e.g., mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Individual or subject includes any subset of the foregoing, e.g., all of the above.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, “altered” can refer to, e.g. a statistically significant change in a level or number (e.g. gene expression level or gene copy number) relative to a reference or a change in a sequence, e.g. at least a single nucleotide change in a nucleic acid sequence relative to a reference (e.g. a wild-type and/or consensus sequence).

As used herein, “normalize” refers to a process of dividing a first value by a second value, e.g. obtaining a level of x per level of y such that the first value is expressed relative to the second. X is typically the thing being measured, e.g. copy number or expression level of NRAS and/or Braf, while y is a reference, e.g. the copy number or expression level of a reference gene. Normalization allows the levels measured in multiple samples and/or reactions to be compared by controlling for, e.g. the level of nucleic acid present in the samples as well as differing efficiencies between reactions.

As used herein, a “portion” refers to a part or fraction of a whole, e.g. a part or fraction of a total molecule. A particular molecule can have multiple portions, e.g. two portions, three portions, four portions, five portions, or more portions.

The term “isolated” or “partially purified” as used herein refers, in the case of a nucleic acid, to a nucleic acid separated from at least one other component (e.g., nucleic acid or polypeptide) that is present with the nucleic acid as found in its natural source and/or that would be present with the nucleic acid when expressed by a cell. A chemically synthesized nucleic acid or one synthesized using in vitro transcription/translation is considered “isolated.”

As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand of a denatured double-stranded DNA. Alternatively, it can be a single-stranded nucleic acid not derived from any double-stranded DNA. In one aspect, a template nucleic acid is DNA. In another aspect, a template is RNA. Suitable nucleic acid molecules include DNA, including genomic DNA and cDNA. Other suitable nucleic acid molecules include RNA, including mRNA, rRNA and tRNA. The nucleic acid molecule can be naturally occurring, as in genomic DNA, or it may be synthetic, i.e., prepared based upon human action, or may be a combination of the two. The nucleic acid molecule can also have certain modifications such as 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA), cholesterol addition, and phosphorothioate backbone as described in US Patent Application 20070213292; and certain ribonucleosides that are linked between the 2′-oxygen and the 4′-carbon atoms with a methylene unit as described in U.S. Pat. No. 6,268,490, wherein both patent and patent application are incorporated herein by reference in their entirety.

The term “gene” means a nucleic acid sequence which is transcribed (DNA) to RNA in vitro or in vivo when operably linked to appropriate regulatory sequences. The gene can include regulatory regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “complementary” refers to the hierarchy of hydrogen-bonded base pair formation preferences between the nucleotide bases G, A, T, C and U, such that when two given polynucleotides or polynucleotide sequences anneal to each other, A pairs with T and G pairs with C in DNA, and G pairs with C and A pairs with U in RNA. As used herein, “substantially complementary” refers to a primer having at least 90% complementarity over the entire length of a primer with a second nucleotide sequence, e.g. 90% complementary, 95% complementary, 98% complementary, 99% complementary, or 100% complementary.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); and Kendrew et al. (eds.), Molecular Biology and Biotechnology: a Comprehensive Desk Reference, published by VCH Publishers, Inc., 1995 (ISBN 1-56081-569-8).

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. An assay for detecting mutations of NRAS and/or BRAF, the assay comprising
   contacting a portion of a nucleic acid sample with a set of primers,
   wherein the set of primers comprises subsets of primer pairs, wherein each primer pair amplifies a NRAS or BRAF sequence comprising a sequence variation;
   performing a PCR amplification regimen comprising cycles of strand separation, primer annealing, and primer extension on a reaction mixture comprising the portion of the sample and the one or more sets of primers;
   detecting the presence or absence of the amplicon for each primer pair;
   wherein the presence of an amplicon indicates the presence of the sequence variation for which that primer pair is specific;
   and wherein one or more of the primers are selected from the group consisting of SEQ ID NOs: 1-39.
2. The assay of paragraph 1, wherein the primer pairs are selected from the group consisting of:
   SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.
3. The assay of any of paragraphs 1-2, wherein one or more sequence variations are point mutations.
4. The assay of any of paragraphs 1-3, wherein the NRAS point mutation is selected from the group consisting of:
   G12D; G12S; G13A; G13C; G13D; G12R; G13V; Q61H1; Q61K; Q61L; Q61R1; and Q61R2.
5. The assay of any of paragraphs 1-4, wherein the BRAF point mutation is selected from the group consisting of: V600D TG/AT; V600E T/A; V600E TG/AA; and V600K GT/AA.
6. The assay of any of paragraphs 1-5, wherein the presence or absence of G12D; G12S; G13A; G13C; G13D; G12R; G13V; Q61H1; Q61K; Q61L; Q61R1; and Q61R2 is detected.
7. The assay of any of paragraphs 1-6, wherein the presence or absence of V600D TG/AT; V600E T/A; V600E TG/AA; and V600K GT/AA is detected.
8. The assay of any of paragraphs 1-7, wherein the nucleic acid sample is prepared from a FFPE tumor sample.
9. The assay of any of paragraphs 1-8, wherein the sample comprises tumor cells from a subject diagnosed with a condition selected from the group consisting of:
   gastric cancer; renal cancer; cholanigoma; lung cancer; brain cancer; cervical cancer;
   colon cancer; head and neck cancer; hepatoma; non-small cell lung cancer;
   melanoma; mesothelioma; multiple myeloma; ovarian cancer; sarcoma; and thyroid cancer.
10. The assay of any of paragraphs 1-9, wherein one or more primers are dual domain primers.
11. The assay of any of paragraphs 1-10, wherein the amplified products from two or more primer pairs of a primer set can be distinguished.
12. The assay of any of paragraphs 1-11, wherein the amplified products from two or more primer pairs of a primer set are distinguished by being of distinct sizes.
13. The assay of any of paragraphs 1-12, wherein the amplified products from two or more primer pairs of a primer set are distinguished by being labeled with different detectable labels.
14. The assay of any of paragraphs 1-13, wherein the primers are present in the reaction mixture at about the concentrations of Example 1.
15. A composition comprising one or more primer pairs selected from the group consisting of molecules having the sequences of:
   SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.
16. The composition of paragraph 15, comprising primer pairs consisting of molecules having the sequences of:
   SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18.
17. The composition of paragraph 15, comprising primer pairs consisting of molecules having the sequences of:
   SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.
18. The composition of any of paragraphs 15-17, wherein the primer pairs are specifically hybridized to target polynucleotides.
19. The composition of any of paragraphs 15-18, wherein at least one member of each primer pair is fluorescently-labelled.
20. A composition comprising fluorescently-labelled amplification products resulting from the amplification of target polynucleotides with one or more primer pairs selected from the group consisting of molecules having the sequences of:
   SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.
21. The composition of any of paragraphs 15-20, further comprising reaction mixture components selected from the group consisting of:
   buffer; dNTPs; and DNA polymerase.
22. The composition of any of paragraphs 15-21, further comprising a nucleic acid sample prepared from a FFPE tumor sample.
23. The composition of any of paragraphs 15-22, further comprising tumor cells from a subject diagnosed with a condition selected from the group consisting of:
   gastric cancer; renal cancer; cholanigoma; lung cancer; brain cancer; cervical cancer;
   colon cancer; head and neck cancer; hepatoma; non-small cell lung cancer;
   melanoma; mesothelioma; multiple myeloma; ovarian cancer; sarcoma; and thyroid cancer.
24. The composition of any of paragraphs 15-23, wherein the primers are present at about the concentrations of Example 1.

### EXAMPLES

### Example 1

Described herein is the development of the NRAS/BRAF point mutation analysis panel, a multiplex PCR assay which can detect the 12 most clinically important NRAS mutations, along with 4 other BRAF mutations using nucleic acid samples, in a single reaction, e.g., on the ICEPlex™ system or similar.

The RAS genes are proto-oncogenes that are frequently mutated in human cancers and are encoded by three ubiquitously expressed genes: HRAS, KRAS, and NRAS. These RAS genes have GTP/GDP binding and GTPase activity and their proteins may be involved in the control of cell growth. RAS proteins exhibit isoform-specific functions and in NRAS, gene mutations which change amino acid residues 12, 13, or 61 activate the potential of the encoded protein to transform cultured cells with implications in a variety of human tumors, particularly cancers of the skin, blood, and lymphoid tissue.

NRAS/BRAF point mutations analysis panel detection primers were designed and then analyzed in silico for primer-primer interaction. Cross-reactivity was determined using the ThermoBlast™ program, wild type cell line gDNA, and synthetic DNA templates. Reaction conditions were optimized on the ICEPlex™ system.

The single-reaction NRAS/BRAF point mutation analysis panel targets the 16 most clinically important mutations in the NRAS and BRAF genes. The assay includes Reference Gene Controls (RGCs) which serve as the DNA fragmentation control and for calculating of a delta Ct to determine mutation status; and calibration controls (C1-3) to determine the size of the PCR amplicons. It is demonstrated herein that the NRAS/BRAF SNP panel is specific to the intended targets.

The NRAS/BRAF point mutation analysis panel detects the mutation status in a single well reaction. The methods and assays described herein permit, e.g., detection of genomic mutations in cancers.

In some embodiments of the aspects described herein, the methods and assays described herein can be performed on an ICEPlex™ system. The ICEPlex™ system is a fully automated real time PCR platform that combines an amplification module (thermocycler) and a detection module module (a capillary electrophoresis cartridge, two solid state lasers with excitation maximum at 488 nm and 639 nm and a spectrophotometer with CCD camera). The ICEPlex™ system generates fluorescently labeled PCR products (amplicons) which are separated based on their different sizes by capillary gel electrophoresis (CE). Amounts of the fluorescent amplicon are monitored in real time by ICEPlex™ system's software that converts the fluorescent signal into amplification curves and calculates cycle thresholds (Cts) for all PCR targets. The combination of PCR and CE permits simultaneous detection and quantification of multiplex targets of as many as, e.g., 48 individual reactions.

Primer Design:

Primers were designed based on the NRAS gene sequence and BRAF gene sequence available from NCBI. Primers were tested in silico using PrimerBlast™ (NCBI). Tag sequences were added to the mutant primers such that the tag should not have homology to the target sequence and have less than 6 nucleotide homology to closely related primers. Primers were tested for primer-primer interaction in multiplex using the Cross-Hyb™ plug-in for the Geneious Pro™ software. Primers were synthesized.

TABLE 1
List of primers designed to generate different sizes of amplicons.

| Primer | | SEQ ID |
|---|---|---|
| Name | Primer Sequence | NO: |
| NRAS- | 5′-TAGGATGGCCAATATTTTTGGTGGTTGGAGCAA-3′ | 1 |
| G12S | | |
| NRAS- | 5′-AAGCTTCGTGATTAATAAATTAATGGTGGTTGGAGCAGA-3′ | 2 |
| G12D | | |
| NRAS- | 5′-ATCGGACTTCTTAAAATAATAAATTAATTGGTTGGAGCAGGTC-3′ | 3 |
| G13R | | |
| NRAS- | 5′-AACTTCTGGGATTTAAAAATATTTTATAATATATGGTTGGAGCAGGTGC | 4 |
| G13A | | |
| NRAS- | 5′-ATCTATATAAAAAAATTAAAAATTAATAAATATTAATAAGGTTGGAGCAG | 5 |
| G13D | GTGA-3′ | |
| NRAS- | 5′-ACCATGGTTTTTATTTATTAATATTATTTATTTAATTTTTATAAGGTTGG | 6 |
| G13V | AGCAGGTGT-3′ | |
| NRAS- | 5′-TGAGTTACCAAATATTTATTTATAATATAAAAATTATTAAAATAATATAG | 7 |
| G13C | GTTGGAGCAGGTT-3′ | |
| NRAS- | /56-FAM/AGACAGGATCAGGTCAGCGG | 8 |
| 12-13- | | |
| Intron- | | |
| R | | |
| NRAS- | 5′-TCAGAAGGACAATAAAAATACTGGATACAGCTGGAA-3′ | 9 |
| Q61K | | |
| NRAS- | 5′-TGGCAGTAGGATAATAATATTAATACTGGATACAGCTGGACT-3′ | 10 |
| Q61L | | |
| NRAS- | 5′-TGTGGAGATTTAATAATTTTAATATAAATACTGGATACAGCTGGACG-3′ | 11 |
| Q61R-1 | | |
| NRAS- | 5′-AGAAGGACCGATTAATTAAAATTTTTATATTTATACTGGATACAGCTGGA | 12 |
| Q61R-2 | CGG-3′ | |
| NRAS- | 5′-AAACGCACAATAATTAATAATAAAATAATTTAATTATATACTGGATACA | 13 |
| Q61H-1 | GCTGGACAC-3′ | |
| NRAS- | /56-FAM/AGATCATCCTTTCAGAGAAAATAATGC | 14 |
| 61- | | |
| Intron- | | |
| R | | |
| V600D- | 5′GCATATCACATTTTGGTCTAGCTACAGAT-3′ | 15 |
| TG/AT-F | | |
| V600E | 5′-CCGCATTTTGGTCTAGCTACAGAG-3′ | 16 |
| -T/A-F | | |
| V600E- | 5′-CATACATAGATACATATAAATTTTGGTCTAGCTACAGAA-3′ | 17 |
| TG/A | | |
| A-F | | |
| V600K- | 5′-CATCATGATCAATTGATTTTGGTCTAGCTACAAA-3′ | 18 |
| GT/A | | |
| A-F | | |

Amplification conditions: PCR reactions were carried out in 1× multiplex PCR buffer (0.3 uM each primer, 0.25×ICEPlex™ calibrator 1, and 1 U of Apta Taq Δexo DNA polymerase. Total reaction volume was 25 uL and reactions were carried out on the ICEPlex™ system. PCR amplification conditions were as follows:

96° C. for 6 minutes, 2 cycles at 54° C. for 45 sec, 72° C. for 45 sec, and 96° C. for 20 sec; 16 cycles at 64° C. for 45 sec, 72° C. for 45 sec, and 98° C. for 5 sec; 28 cycles at 64° C. for 45 sec, 72° C. C for 220 sec, 96° C. for 10 sec.

TABLE 2
Alterations detected by the primers of Table 1
The ICEPlex NRAS/BRAF Panel detects:

| | Amino Acid |
|---|---|
| CDS Mutation | Residue Change |
| NRAS c.35 G > A | G12D |
| NRAS c.34 G > A | G12S |
| NRAS c.38 G > C | G13A |
| NRAS c.38 G > A | G13D |
| NRAS c.37G > C | G13R |
| NRAS c.38 G > T | G13V |
| NRAS c.37 G > T | G13C |
| NRAS c.183 A > C(H1) | Q61H |
| NRAS c.182 A > G (R1) | Q61R |
| NRAS c.182_183AA > GG (R2) | Q61R |
| NRAS c.182 A > T | Q61L |
| NRAS c. 181 C > A | Q61K |
| BRAF c.1799 1800 TG > AT | V600D |
| BRAF c.1799 T > A | V600E |
| BRAF c.1799 1800 TG/AA | V600E |
| BRAF c.1798 1799 GT > AA | V600K |

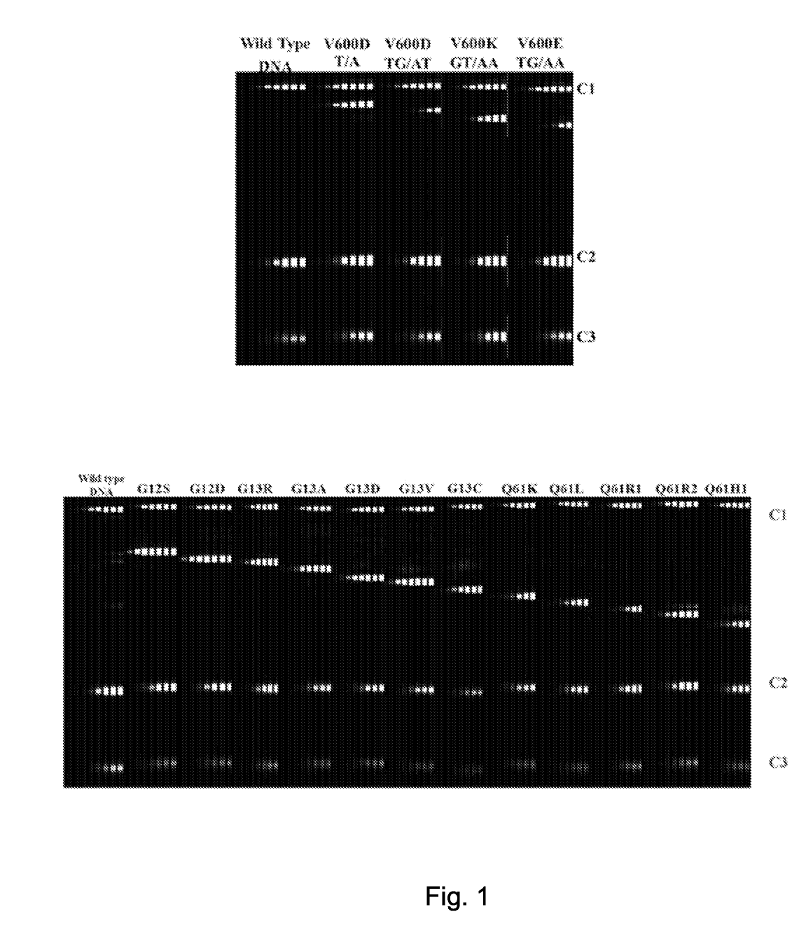

The assay is capable of simultaneously detecting and differentiating 4 BRAF targets and 12 NRAS targets (FIG. 1). Two different dyes (FAM and TYE) were used. Calibrator controls were used to size the targets in the assay.

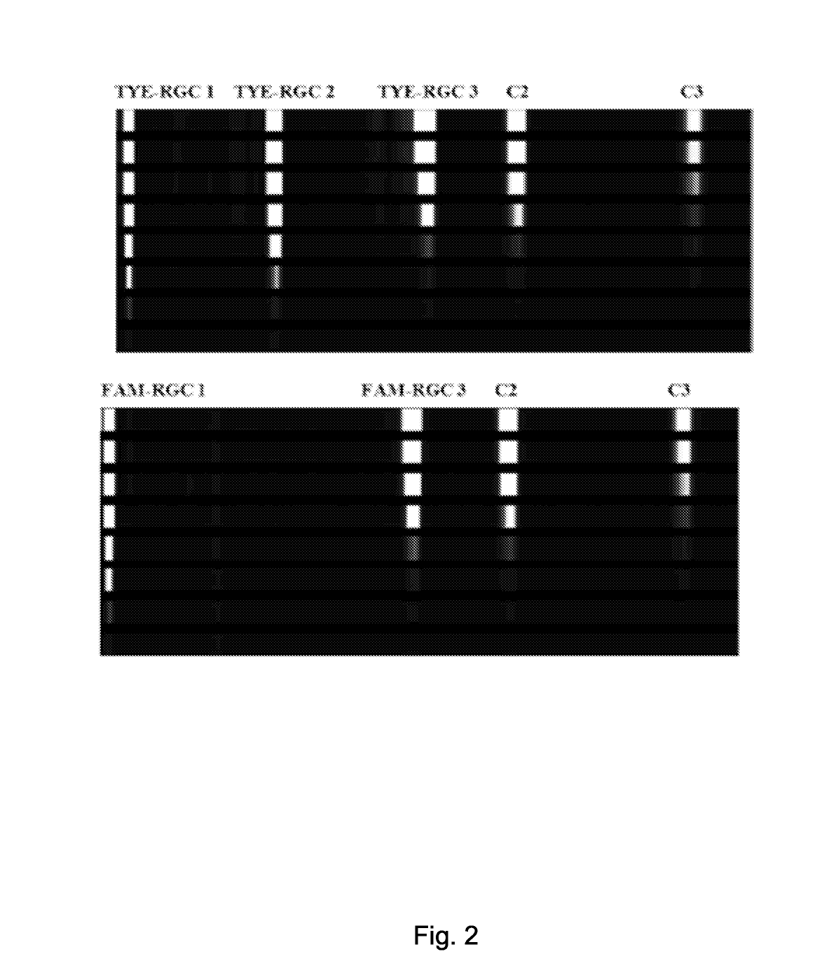

Reference gene controls to permit internal DNA fragmentation controls were developed. These controls permit determination of the quality of the DNA starting material as well as for calculating the delta Ct. Three primer pairs amplify the reference gene controls on the NRAS gene. 3 targets—TYE-RGC1, 2, and 3 are detected in the TYE channel and 2 targets are detected in the FAM channel (FAM-RGC1, and 2) (FIG. 2). The reference gene controls can be included in a single multiplex reaction with the primers of Table 1.

TABLE 3
NRAS/BRAF Point mutation analysis panel, results from cross-
rectivity study. High specificity was demonstrated, with
cross-reactivity observed for only two targets.

| Synthetic template added | | |
|---|---|---|
| to the multiplex PCR | Target | Crossreative on other |
| reactions | detected | assay targets |
| G12D | Detected | Not detected |
| G12S | Detected | Not detected |
| G13A | Detected | Not detected |
| G13C | Detected | Not detected |
| G13D | Detected | Not detected |
| G13R | Detected | Not detected |
| G13V | Detected | Not detected |
| Q61H1 | Detected | Detected at delayed Cts |
| Q61K | Detected | Not detected |
| Q61L | Detected | Not detected |
| Q61R1 | Detected | Not detected |
| Q61R2 | Detected | Detected at delayed Cts |
| V600D TG/AT | Detected | Not detected |
| V600E T/A | Detected | Not detected |
| V600E TG/AA | Detected | Not detected |
| V600K GT/AA | Detected | Not detected |
| Wild type genomic DNA | N/A | Not detected |
| from cell line K562 | | |
| (Negative control) | | |

Described herein is a NRAS/BRAF point mutation analysis panel developed for the detection of point mutations in the NRAS and BRAF oncogene biomarkers. The high multiplex NRAS/BRAF point mutation analysis panel detects no only all target NRAS/BRAF mutations, but also reference gene controls for DNA fragmentation and delta Ct calculation and calibration controls for sizing. The results from the studies described herein demonstrate that the NRAS/BRAF point mutation analysis panel is highly specific for discrimination of all the targeted NRAS/BRAF mutations in a high multiplex single-tube format.

### Example 2

TABLE 4
Alternative set of NRAS primers compatible with the conditions described
in Example 1, including the BRAF primers presented in Table 1.

| | | Core | Actual | SEQ |
|---|---|---|---|---|
| Target | | Amplicon | Size on | ID |
| Name | Primer Sequences | Size | ModaPlex | NO |
| NRAS- | TAAATTTTAATTTTAAAACCATGGTTTCAA | 55 | 135 | 19 |
| G12D- | GCTTCATAAATTAATGGTGGTTGGAGCAG | | | |
| Primary- | A | | | |
| cDNA | | | | |
| NRAS- | ATAAATGGTATTTTAATTTTAAAACCATGG | 55 | 139 | 20 |
| G12S- | TTTCTAGGATGGCTTTGGTGGTTGGAGCAA | | | |
| Primary- | | | | |
| cDNA | | | | |
| NRAS- | TAACTTCTGGGATTAAATATTTTATAATAT | 55 | 119 | 21 |
| G13A- | ATGGTTGGAGCAGGTGC | | | |
| Primary- | | | | |
| cDNA | | | | |
| NRAS- | ATATATTGAGTTACCAAATATTTATTTATA | 55 | 144 | 22 |
| G13C- | ATATAAAAATTATTAAAATAATATAGGTT | | | |
| Primary- | GGAGCAGGTT | | | |
| cDNA | | | | |
| NRAS- | GCTATATAAAATTAAAAATTAATAAATAT | 55 | 123 | 23 |
| G13D- | TAATAAGGTTGGAGCAGGTGA | | | |
| Primary- | | | | |
| cDNA | | | | |
| NRAS- | ATAAAACCATGGTTTATCGGACTTCAATA | 55 | 127 | 24 |
| G13R- | ATAAATTAATTGGTTGGAGCAGGTC | | | |
| Primary- | | | | |
| cDNA | | | | |
| NRAS- | ATACCATGGTTTTTATTTATTAATATTATTT | 55 | 131 | 25 |
| G13V- | ATTTAATTTTTATAAGGTTGGAGCAGGTGT | | | |
| Primary- | | | | |
| cDNA | | | | |
| NRAS- | ATATTAAAACGCACAATAATTAATAATAA | 72 | 138 | 26 |
| Q61H-1- | AATAATTTAATTATATACTGGATACAGCTG | | | |
| Primary- | GACAC | | | |
| cDNA | | | | |
| NRAS- | TTATTAAAACTTATATCGAAATAATATTAT | 72 | 126 | 27 |
| Q61H-2- | ATTATAATTTATTTATTAATATACTGGATA | | | |
| Primary- | CAGCTGGACAT | | | |
| cDNA | | | | |
| NRAS- | TATCAGAAGGACAATAAAAATACTGGATA | 72 | 142 | 28 |
| Q61K- | CAGCTGGAA | | | |
| Primary- | | | | |
| cDNA | | | | |
| NRAS- | AATTGGCAGTAGGATAATAATATTAATAC | 72 | 122 | 29 |
| Q61L- | TGGATACAGCTGGACT | | | |
| Primary- | | | | |
| cDNA | | | | |
| NRAS- | TAAATTGTGGAGATTTAATAATTTTAATAT | 72 | 130 | 30 |
| Q61R-1- | AAATACTGGATACAGCTGGACG | | | |
| Primary- | | | | |
| cDNA | | | | |
| NRAS- | TAAATTAGAAGGACCGATTAATTAAAATT | 72 | 134 | 31 |
| Q61R-2- | TTTATATTTATACTGGATACAGCTGGACGG | | | |
| Primary- | | | | |
| cDNA | | | | |
| NRAS | /56- | | | 32 |
| 61_F_c | FAM/TAGGATGGCCTTATTAATATTAGAGG | | | |
| DNA | AAGCCTTCGCCTGTC | | | |
| NRAS_ | /5TYE665/TAGGATGGCCTAATTAATAATAA | | | 33 |
| 12&13_ | AATAATTTAATTATTGGATTAGCTGGATTG | | | |
| F_cDNA | TCAGTGC | | | |
| NRAS- | AACAGCAGCCATTACCAGACCCATCCATT | 89 | 118 | 34 |
| Ref-3 F | CCCGTG | | | |
| NRAS- | AATTATATTTTGGAAGAACCCAGGGCAGA | 149 | 170 | 35 |
| Ref-6 F | A | | | |
| NRAS- | CTTTGACAGATTTATGGAATCCCACACGG | 181 | 220 | 36 |
| Ref-10 F | GACGTTTCAAT | | | |
| NRAS- | /5TYE665/ATATATTTTATCACCGCCTGGTT | | | 37 |
| Ref-3 R | ACTGTGTCCTG | | | |
| NRAS- | /5TYE665/AGTTGAGATATAGCTCCTGCTAC | | | 38 |
| Ref-6 R | TTCCCCAG | | | |
| NRAS- | /5TYE665/ACAGTAATAACTTACATAATCCC | | | 39 |
| Ref-10 R | CCTTGTCGTTTGAGGT | | | |

The primers of Table 4 are compatible with cDNA templates as well as genomic DNA.

1. An assay for detecting mutations of NRAS and/or BRAF, the assay comprising
contacting a portion of a nucleic acid sample with a set of primers,
wherein the set of primers comprises subsets of primer pairs, wherein each primer pair amplifies a NRAS or BRAF sequence comprising a sequence variation;
performing a PCR amplification regimen comprising cycles of strand separation, primer annealing, and primer extension on a reaction mixture comprising the portion of the sample and the one or more sets of primers;
detecting the presence or absence of the amplicon for each primer pair;
wherein the presence of an amplicon indicates the presence of the sequence variation for which that primer pair is specific;
and wherein one or more of the primers are selected from the group consisting of SEQ ID NOs: 1-39.

2. The assay of claim 1, wherein the primer pairs are selected from the group consisting of:
SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

3. The assay of claim 1, wherein one or more sequence variations are point mutations.

4. The assay of claim 3, wherein the NRAS point mutation is selected from the group consisting of:
G12D; G12S; G13A; G13C; G13D; G12R; G13V; Q61H1; Q61K; Q61L; Q61R1; and Q61R2.

5. The assay of claim 3, wherein the BRAF point mutation is selected from the group consisting of:
V600D TG/AT; V600E T/A; V600E TG/AA; and V600K GT/AA.

6. The assay of claim 4, wherein the presence or absence of G12D; G12S; G13A; G13C; G13D; G12R; G13V; Q61H1; Q61K; Q61L; Q61R1; and Q61R2 is detected.

7. The assay of claim 5, wherein the presence or absence of V600D TG/AT; V600E T/A; V600E TG/AA; and V600K GT/AA is detected.

8. The assay of claim 1, wherein the nucleic acid sample is prepared from a FFPE tumor sample.

9. The assay of claim 1, wherein the sample comprises tumor cells from a subject diagnosed with a condition selected from the group consisting of:
gastric cancer; renal cancer; cholanigoma; lung cancer; brain cancer; cervical cancer;
colon cancer; head and neck cancer; hepatoma; non-small cell lung cancer; melanoma;
mesothelioma; multiple myeloma; ovarian cancer; sarcoma; and thyroid cancer.

10. The assay of claim 1, wherein one or more primers are dual domain primers.

11. (canceled)

12. The assay of claim 1, wherein the amplified products from two or more primer pairs of a primer set are distinguished by being of distinct sizes or by being labeled with different detectable labels.

13. (canceled)

14. (canceled)

15. A composition comprising one or more primer pairs selected from the group consisting of molecules having the sequences of:
SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

16. The composition of claim 15, comprising primer pairs consisting of molecules having the sequences of:
SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18.

17. The composition of claim 15, comprising primer pairs consisting of molecules having the sequences of:
SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

18. The composition of claim 15, wherein the primer pairs are specifically hybridized to target polynucleotides.

19. The composition of claim 15, wherein at least one member of each primer pair is fluorescently-labelled.

20. A composition comprising fluorescently-labelled amplification products resulting from the amplification of target polynucleotides with one or more primer pairs selected from the group consisting of molecules having the sequences of:
SEQ ID NO:s 1 and 8; SEQ ID NO:s 2 and 8; SEQ ID NO:s 3 and 8; SEQ ID NO:s 4 and 8; SEQ ID NO:s 5 and 8; SEQ ID NO:s 6 and 8; SEQ ID NO:s 7 and 8; SEQ ID NO:s 9 and 14; SEQ ID NO:s 10 and 14; SEQ ID NO:s 11 and 14; SEQ ID NO:s 12 and 14; SEQ ID NO:s 13 and 14; SEQ ID NO:s 15 and 18; SEQ ID NO:s 16 and 18; SEQ ID NO:s 17 and 18; SEQ ID NO:s 19 and 33; SEQ ID NO:s 20 and 33; SEQ ID NO:s 21 and 33; SEQ ID NO:s 22 and 33; SEQ ID NO:s 23 and 33; SEQ ID NO:s 24 and 33; SEQ ID NO:s 25 and 33; SEQ ID NO:s 26 and 32; SEQ ID NO:s 27 and 32; SEQ ID NO:s 28 and 32; SEQ ID NO:s 29 and 32; SEQ ID NO:s 30 and 32; SEQ ID NO:s 31 and 32; SEQ ID NO:s 34 and 37; SEQ ID NO:s 35 and 38; and SEQ ID NO:s 36 and 39.

21. The composition of claim 15, further comprising reaction mixture components selected from the group consisting of:
buffer; dNTPs; and DNA polymerase.

22. The composition of claim 15, further comprising a nucleic acid sample prepared from a FFPE tumor sample.

23. The composition of claim 15, further comprising tumor cells from a subject diagnosed with a condition selected from the group consisting of:
gastric cancer; renal cancer; cholanigoma; lung cancer; brain cancer; cervical cancer;
colon cancer; head and neck cancer; hepatoma; non-small cell lung cancer; melanoma;
mesothelioma; multiple myeloma; ovarian cancer; sarcoma; and thyroid cancer.

24. (canceled)
